(19) United States
(12) Patent Application Publication
(10) Pub. No.: US 2012/0245100 A1
(43) Pub. Date: Sep. 27, 2012

(54) PLASMID FOR EXPRESSION OF TRANSGENES IN PLANTS

(76) Inventors: Brennan Winkler, South Jordan, UT (US); Ellen Peterson, Sandy, UT (US)

(21) Appl. No.: 13/427,851

(22) Filed: Mar. 22, 2012

Related U.S. Application Data
(60) Provisional application No. 61/466,303, filed on Mar. 22, 2011.

(51) Int. Cl. C12N 15/82; A61K 38/16; A61K 35/00; A01H 5/00

(52) U.S. Cl. 514/19.3; 435/320.1; 800/298; 800/320.1; 800/320.2; 800/313; 800/317.4

(57) ABSTRACT

A plasmid for use in transferring a chicken anemia virus apoptin coding sequence into a plant includes a binary vector suitable for transferring DNA from Agrobacterium tumefaciens to a selected plant, upstream and downstream homologous regions for targeting transfer of the DNA to a specified location in the selected plant genome, a chicken anemia virus apoptin coding sequence operationally configured adjacent to selected plant promoter and terminator segments, and an antibiotic marker coding sequence operationally configured adjacent to selected plant promoter and terminator segments. Transfer of DNA into a plant, such as Arabidopsis thaliana, results in a plant that expresses apoptin. Cancer treatment can include ingesting plant parts containing expressed apoptin. Apoptin for use as a medicine or vaccine can be obtained from the modified plant.

### CROSS-REFERENCE TO RELATED APPLICATIONS

This application claims the benefit of U.S. Provisional Application No. 61/466,303, filed Mar. 22, 2011, which is hereby incorporated by reference herein in its entirety, including but not limited to those portions that specifically appear hereinafter, the incorporation by reference being made with the following exception: In the event that any portion of the above-referenced provisional application is inconsistent with this application, this application supercedes the above-referenced provisional application.

### STATEMENT REGARDING FEDERALLY SPONSORED RESEARCH OR DEVELOPMENT

Not applicable.

### BACKGROUND OF THE INVENTION

This invention relates to a plasmid for transferring foreign DNA into a plant. An illustrative embodiment of this invention relates to a plasmid containing a chicken anemia virus apoptin coding sequence for transfer of the apoptin coding sequence into a plant, such as Arabidopsis thaliana. More particularly, this illustrative embodiment of the invention relates to insertion of a chicken anemia virus apoptin coding sequence into A. thaliana by Agrobacterium-mediated gene transfer.

Apoptin is a protein composed of 121 amino acids and is found in the chicken anemia virus. It has been shown to reduce tumor size and kill cancerous cells. Results have shown that over 70 different human cancer cell lines transfected with the apoptin gene are affected by its protein. Research also shows that apoptin does not affect normal human epithelial cells and other tested human cells. Apoptin has been shown to affect cancerous cells even when it is not being produced. After one cell is transfected with apoptin, the protein is secreted and affects other cancerous cells.

### BRIEF SUMMARY OF THE INVENTION

An illustrative embodiment of the invention comprises a plasmid comprising segments operationally configured adjacent to each other in the following order:

a binary vector comprising a broad-host-range replication origin, a gene encoding antibiotic resistance in bacteria, T-DNA border repeats configured for mediating transfer of DNA from Agrobacterium tumefaciens into a selected plant, and a multiple cloning site positioned between the T-DNA border repeats;

an upstream homologous region for targeting, together with a downstream homologous region, incorporation of DNA transferred from Agrobacterium tumefaciens into the genome of the selected plant;

a first promoter for promoting transcription of a first adjacent sequence;

the first adjacent sequence;

a first terminator for signaling termination of transcription of the first adjacent sequence;

a second promoter for promoting transcription of a second adjacent sequence;

the second adjacent sequence;

a second terminator for signaling termination of transcription of the second adjacent sequence; and

the downstream homologous region for targeting, together with the upstream homologous region, incorporation of DNA transferred from Agrobacterium tumefaciens into the genome of the selected plant. Illustratively, the first adjacent sequence can comprise a chicken anemia virus apoptin coding sequence. Further, the binary vector can comprise pCB301. Still further, the selected plant can comprise Arabidopsis thaliana, and the upstream homologous region and the downstream homologous region can comprise Arabidopsis thaliana genomic DNA. Also, the first promoter and the second promoter can comprise an Arabidopsis thaliana SUC2 promoter. By way of further illustration, the first terminator and the second terminator can comprise a nopaline synthase (NOS) terminator. Moreover, the second adjacent sequence can comprise a marker for antibiotic resistance in the selected plant, such as gentamicin resistance.

Another illustrative embodiment of the invention comprises the plasmid described in the previous paragraph wherein the selected plant comprises Arabidopsis thaliana, the binary vector comprises pCB301, upstream homologous region comprises Arabidopsis thaliana genomic DNA, the first promoter comprises an Arabidopsis thaliana SUC2 promoter, the first adjacent sequence comprises a chicken anemia virus apoptin coding sequence, the first terminator comprises a nopaline synthase terminator, the second promoter comprises an Arabidopsis thaliana SUC2 promoter, the second adjacent sequence comprises a gentamicin resistance marker, the second terminator comprises a nopaline synthase terminator, and the downstream homologous region comprises Arabidopsis thaliana genomic DNA.

Still another illustrative embodiment of the invention comprises a plant expressing chicken anemia virus apoptin. For example, the plant can comprise Arabidopsis thaliana. In other illustrative embodiments of the invention, the plant can comprise pea, corn, rice, sweet potato, rhubarb, chick pea, or tomato.

Further yet, another illustrative embodiment of the invention comprises a method for treating cancer comprising administering a plant part comprising apoptin to a patient in need thereof.

### BRIEF DESCRIPTION OF THE SEVERAL VIEWS OF THE DRAWINGS

FIG. 1 shows a map of the pSArPP plasmid according to the present invention.

FIG. 2 shows a schematic diagram of plasmid construction with the one-step isothermal reaction.

### DETAILED DESCRIPTION

Before the present compositions and methods relating to a plasmid for inserting a transgene into a plant are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The publications and other reference materials referred to herein to describe the background of the invention and to provide additional detail regarding its practice are hereby incorporated by reference. The references discussed herein are provided solely for their disclosure prior to the filing date of the present application. Nothing herein is to be construed as an admission that the inventors are not entitled to antedate such disclosure by virtue of prior invention.

It must be noted that, as used in this specification and the appended claims, the singular forms “a,” “an,” and “the” include plural referents unless the context clearly dictates otherwise.

Unless defined otherwise, all technical and scientific terms used herein have the same meanings as commonly understood by one of ordinary skill in the art to which this invention belongs.

In describing and claiming the present invention, the following terminology will be used in accordance with the definitions set out below.

As used herein, “comprising,” “including,” “containing,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.”

As used herein, “consisting of” and grammatical equivalents thereof exclude any element, step, or ingredient not specified in the claim.

As used herein, “consisting essentially of” and grammatical equivalents thereof limit the scope of a claim to the specified materials or steps and those that do not materially affect the basic and novel characteristic or characteristics of the claimed invention.

As used herein, “transgene” means a foreign DNA that is inserted into a selected plant. For example, DNA encoding chicken anemia virus apoptin would be a transgene for insertion into A. thaliana. As used herein, the term “transgene” carries no connotation associated with the term “gene,” such as the presence of a promoter, enhancer, transcription factor binding sites, terminator, or the like.

### Plasmid Construction

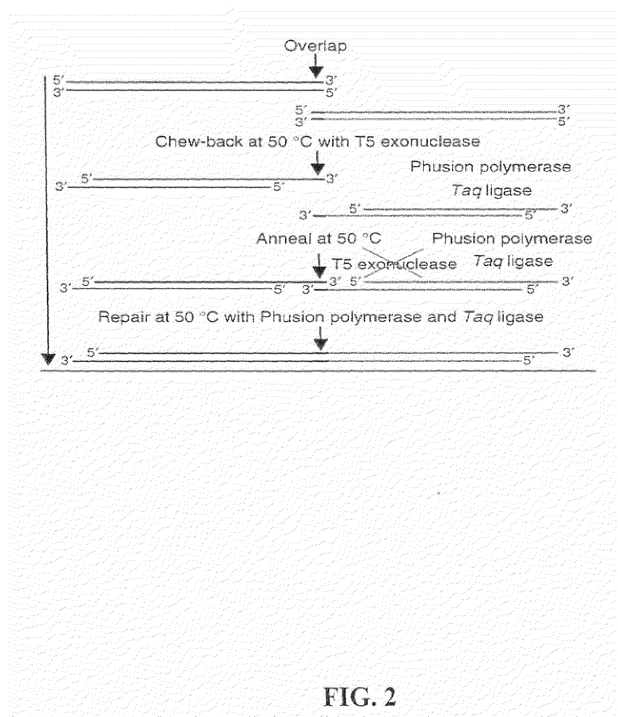

To obtain the necessary segments for construction of the pSArPP plasmid, DNA segments were amplified using PCR and then were connected together with a one-step isothermal reaction (FIG. 2). All of the primers contain an overlap of the previous and following plasmid sequences to make them specific to each other and to the order of the plasmid. After obtaining the selected fragments by PCR, a one-step isothermal reaction was used to construct the plasmid (all PCR reactions were carried out with: Phusion® taq, Phire® taq, PrimeSTAR® taq, PHIRE II® taq, and PHUSION® Hot Start Flex taq). The one-step isothermal reaction uses T5 exonuclease, Taq ligase, and Phusion® polymerase. As shown in FIG. 2, after the T5 exonuclease cuts back the ends of the PCR products, which it denatures within 10-15 minutes, the sticky ends of the products find each other and anneal spontaneously, the PHUSION® polymerase then fills in any gaps created by the exonuclease, and Taq ligase then reconstitutes the phosphodiester bonds between adjacent non-connected nucleotides. The reaction is typically run in a thermocycler for an hour at 50° C. The exonuclease denatures throughout the process, which allows the Taq ligase and Phusion® polymerase to put together the entire plasmid. All the PCR products are included in the same reaction to create the plasmid in one step.

The construction of pCB301 is described in X. Chengbin et al., A mini binary vector series for plant transformation, 40 Plant Molec. Biol. 711-717 (1999) (nucleotide sequence is available in EMBL, GenBank, and DDBJ nucleotide sequence databases as accession number AF 139061). The plasmid pCB301 is a small (3.5 kb) binary vector and is ready for accepting DNA fragments that are to be transferred into the plant genome. It contains the broad-host-range RK2 replication origin and the nptIII gene, which confers kanamycin resistance in Escherichia coli and other bacteria. It also contains T-DNA border repeats enclosing a multiple cloning site (MCS). Thus, DNA inserted between the T-DNA border repeats is transferred into a plant genome upon transfection. The plasmid pCB301 is incapable of conjugal transfer to Agrobacterium, but transfer to Agrobacterium can be readily achieved by direct transformation, such as with the well known electroporation or freeze-thaw methods. The plasmid pCB301 is stable in both E. coli and A. tumefaciens. The forward and reverse primers for amplification of pCB301 were SEQ ID NO:1 and SEQ ID NO:2.

The upstream and downstream homologous regions are sequences taken from sections of the plant to be modified, such as Arabidopsis thaliana. The presence of these homologous regions in the plasmid allows for gene targeting in the transformed plant, as opposed to insertion at a random location. In the plasmid, the homologous regions lie on either side of the sequences that are to be inserted into the plant genome. These homologous regions bind a selected section of the plant genome such that the heterologous DNA is inserted specifically. This allows the DNA to be inserted into a selected reading frame such that the heterologous DNA is expressible, instead of being rendered inoperable by random insertion. The forward and reverse primers for amplification of the A. thaliana upstream homologous region are SEQ ID NO:3 and SEQ ID NO:4. Thus, the upstream homologous region is defined by the PCR product of these two primers. The forward and reverse primers for amplification of the A. thaliana downstream homologous region are SEQ ID NO:15 and SEQ ID NO:16. Accordingly, the downstream homologous region is defined by the PCR product of these two primers.

The SUC2 promoter is a promoter from the Arabidopsis thaliana SUC2 gene, which encodes a plasma-membrane sucrose-proton symporter. The SUC2 symporter is responsible for transporting sucrose throughout the entire plant. The promoter also promotes gene expression throughout the entire plant. In the plasmid construct, SUC2 promoters were placed immediately upstream of both the apoptin coding sequence and the gentamicin coding sequence. The forward and reverse primers for the SUC2 promoter upstream of the apoptin coding sequence were SEQ ID NO:5 and SEQ ID NO:6, respectively. The forward and reverse primers for the SUC2 promoter upstream of the gentamicin coding sequence were SEQ ID NO:11 and SEQ ID NO:12, respectively.

The Nos (nopaline synthase) terminator is a stop signal for gene transcription. It is placed downstream of the coding sequence that is to be expressed, in this case, the apoptin coding sequence. Nos terminators were placed immediately downstream of both the apoptin coding sequence and the gentamicin coding sequence. The forward and reverse primers for the Nos terminator downstream of the apoptin coding sequence were SEQ ID NO:9 and SEQ ID NO:10, respectively. The forward and reverse primers for the Nos terminator downstream of and fused to the gentamicin coding sequence were SEQ ID NO:13 and SEQ ID NO:14, respectively.

The apoptin coding sequence encodes apoptin, a protein from chicken anemia virus that is composed of 121 amino acid residues. As mentioned above, apoptin has been shown to reduce tumor size and kill cancer cells. Results have shown that more than 70 different human cancer cell lines transfected with the apoptin gene are affected by its protein. Research also shows that apoptin does not affect normal human epithelial cells and other tested human cells. Apoptin has been shown to affect cancer cells even when apoptin is not being produced. After one cell is transfected with apoptin, the protein is secreted and affects other cancer cells. The forward and reverse primers for the apoptin coding sequence were SEQ ID NO:7 and SEQ ID NO:8.

The gentamicin resistance coding sequence is used to for positive selection of clones after plant modification. When gentamicin resistance is expressed, then the plants have been modified and it is likely that apoptin is being produced throughout the plant. The forward and reverse primers for the gentamicin coding sequence were SEQ ID NO:13 and SEQ ID NO:14.

Transformation of E. coli and Agrobacterium

Agrobacterium cells are transformed with the plasmid described above using the freeze thaw method or electroporation. Then, restriction mapping is performed with HindIII or HhaI restriction enzyme to confirm the plasmid construction. The correct construction of the pSArPP plasmid can also be determined by PCR amplification of the segment junctions, namely SEQ ID NO:18 through SEQ ID NO:33.

Modification of Arabidopsis thaliana with the Apoptin Gene

Arabidopsis thaliana is modified by Agrobacterium-mediated gene transfer. Leaf, floral, or stem dip methods can be used for modification, according to methods well known in the art. When the Agrobacterium is introduced to plant leaves, it interacts with the plant cells and creates a passageway for the linearized plasmid, contained in the Ti region of the bacterium DNA, to get into the plant genome. Agrobacterium is able to do this because of the virulence genes it contains. The vector in the plasmid contains DNA sequences that promote the Agrobacterium to cut the homologous sequence out and transfer it into the plant. The virulence genes interact with the plant and create a pathway for the linearized plasmid to be inserted into the genomic, and chloroplast DNA and the gene is then translated by the plant.

Protein expression is tested by treating the plants with an antibiotic. When the plants are producing functional proteins from the linearized plasmid, they are not affected by the treatments. The plants that have positive results are then tested for apoptin protein expression by western blotting and DNA analysis. Western blotting is performed with polyacrylamide gel electrophoresis followed by protein detection using apoptin antibodies. Plant DNA extraction and PCR are used to verify the presence of the linearized plasmid in the Arabidopsis thaliana genome.

### Example 1

### Amplification of the pCB301 Plasmid Segment

The pCB301 plasmid segment of the pSArPP plasmid was synthesized by PCR using the forward and reverse primers SEQ ID NO:1 and SEQ ID NO:2, respectively. The reaction was carried out in a 20 μl volume containing PHIRE II® PCR reaction buffer, the four deoxynucleoside triphosphates (0.2 mM), the forward primer (SEQ ID NO:1; 0.5 μM), the reverse primer (SEQ ID NO:2; 0.5 μM), pCB301 DNA extracted from E. coli, and PHIRE® Hot Start polymerase II.

The amplified DNA was assayed by gel electrophoresis. A band of about 3.5 kb was detected, thus confirming the amplification of the pCB301 plasmid segment.

### Example 2

### Amplification of A. thaliana Upstream Homologous Target Segment

An A. thaliana upstream homologous target segment was amplified by PCR according to the procedure of Example 1 except that A. thaliana genomic DNA was substituted for pCB301 DNA as the amplification template, and primers SEQ ID NO:3 and SEQ ID NO:4 were substituted for SEQ ID NO:1 and SEQ ID NO:2, respectively. The resulting amplified DNA was assayed by gel electrophoresis, and a band of about 6 kb was produced, confirming the amplification of the template DNA.

### Example 3

### Amplification of SUC2 Promoter Segments

Two versions of the SUC2 promoter segments were amplified according to the procedure of Example 1 except for the changes noted next. A. thaliana genomic DNA was substituted for pCB301 DNA as the amplification template. In the version of the SUC2 promoter segment to be placed upstream of the apoptin coding sequence, primers SEQ ID NO:5 and SEQ ID NO:6 were substituted for primers SEQ ID NO:1 and SEQ ID NO:2, respectively. In the version of the SUC2 promoter segment to be placed upstream of the gentamicin coding sequence, primers SEQ ID NO:11 and SEQ ID NO:12 were substituted for primers SEQ ID NO:1 and SEQ ID NO:2, respectively.

The amplified DNA was assayed by gel electrophoresis, and in each case bands of about 2100 bp were detected, thus confirming the amplification of the SUC2 promoter segments.

### Example 4

### Amplification of the Apoptin Coding Sequence Segment The chicken anemia virus apoptin coding sequence (SEQ ID NO:17) was cloned in a pIDTSMART vector by Integrated DNA Technologies (Coralville, Iowa) after custom order. The apoptin coding sequence segment was amplified by PCR according to the procedure of Example 1 except that the custom plasmid was substituted for pCB301 DNA as the template, primers SEQ ID NO:7 and SEQ ID NO:8 were substituted for primers SEQ ID NO:1 and SEQ ID NO:2, respectively, and PHUSION® reaction buffer and PHUSION® DNA polymerase (New England BioLabs, Ipswich, Mass.) were substituted for PHIRE® reaction buffer and polymerase, respectively.

The amplified DNA was assayed by gel electrophoresis, and a band of about 360 bp was detected, thus confirming the amplification of the chicken anemia virus apoptin coding sequence segment.

### Example 5

### Amplification of Nos Terminator Segment

The gentamicin coding sequence and a Nos terminator segment were cloned in a pIDT vector by Integrated DNA Technologies upon custom order. The sequence of the fused gentamicin coding sequence and the NOS terminator is SEQ ID NO:34. The Nos terminator segment to be placed downstream of the apoptin coding sequence was amplified according to the procedure of Example 1 except the custom gentamicin-Nos plasmid was substituted for the pCB301 DNA as the amplification template, and primers SEQ ID NO:9 and SEQ ID NO:10 were substituted for primers SEQ ID NO:1 and SEQ ID NO:2, respectively. The amplified DNA was assayed by gel electrophoresis, and a band of the expected size (about 260 bp) was detected, thus confirming the amplification of the Nos terminator segment.

### Example 6

### Amplification of Gentamicin Coding Sequence-NOS Terminator Segment

The gentamicin coding sequence segment was amplified by PCR according to the procedure of Example 1 except that the custom gentamicin-Nos plasmid described in Example 5 was substituted for pCB301 DNA as the template, and primers SEQ ID NO:13 and SEQ ID NO:14 were substituted for primers SEQ ID NO:1 and SEQ ID NO:2, respectively. Use of these primers resulted in amplification of a fused gentamicin coding sequence-Nos terminator amplification product.

The amplified DNA was assayed by gel electrophoresis, and a band of about 420 bp was detected, thus confirming the amplification of the fused gentamicin coding sequence-Nos terminator segment.

### Example 7

### Amplification of A. thaliana Downstream Homologous Target Segment

An A. thaliana downstream homologous target segment was amplified by PCR according to the procedure of Example 1 except that A. thaliana genomic DNA was substituted for pCB301 DNA as the amplification template, and primers SEQ ID NO:15 and SEQ ID NO:16 were substituted for SEQ ID NO:1 and SEQ ID NO:2, respectively. The resulting amplified DNA was assayed by gel electrophoresis, and a band of about 6 kb was produced, confirming the amplification of the template DNA.

### Example 8

### Construction of pSArPP Plasmid

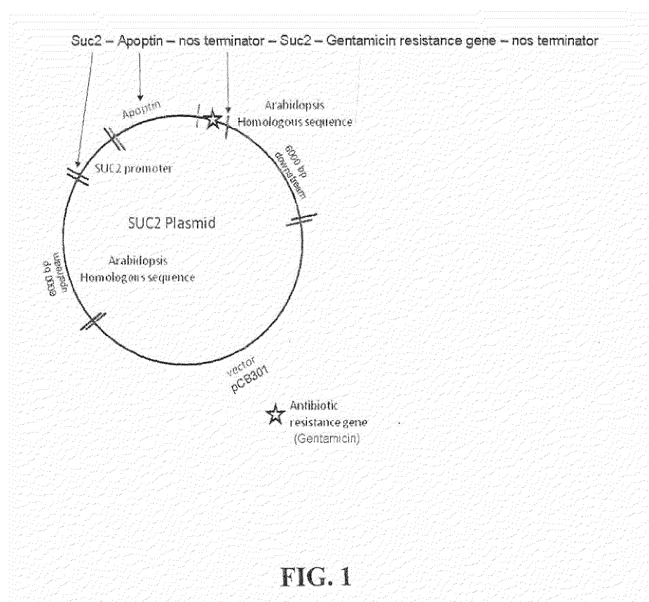

The pSArPP plasmid was constructed by assembling the pCB301 vector, upstream and downstream homologous regions from the genomic target in A. thaliana, the SUC2 promoter, the Nos terminator, a gentamicin resistance coding sequence, and the apoptin coding sequence, as shown in FIG. 1. Each of these segments of the pSArPP plasmid was constructed by polymerase chain reaction (PCR), as described above in Examples 1-7.

An isothermal reaction was carried out by combining all nine PCR fragments, isothermal reaction buffer (500 mM Tris-HCl, pH 7.5, 50 mM MgCl2, 1 nM of each of the four deoxynucleoside triphosphates, 50 mM dithiothreitol, 25% PEG-8000, 5 mM NAD), T5 exonuclease, PHUSION® DNA polymerase, and Taq DNA ligase. The reaction was carried out for 1 hour at 50° C.

Results were assayed by PCR and gel electrophoresis to determine if all of the expected segment junctions could be amplified, which would show that all of the segments of the plasmid came together in the correct order as a result of the isothermal reaction. The primers for detecting the correct joining of the pCB301 and A. thaliana upstream homologous region were SEQ NO:18 and SEQ ID NO:19. The primers for detecting the correct joining of the A. thaliana upstream homologous region and the SUC2 promoter were SEQ ID NO:20 and SEQ ID NO:21. The primers for detecting the correct joining of the SUC2 promoter and the apoptin coding sequence were SEQ ID NO:22 and SEQ ID NO:23. The primers for detecting the correct joining of the apoptin coding sequence and the NOS terminator were SEQ ID NO:24 and SEQ ID NO:25. The primers for detecting the correct joining of the NOS terminator and the SUC2 promoter were SEQ ID NO:26 and SEQ ID NO:27. The primers for detecting the correct joining of the SUC2 promoter and the gentamicin-NOS terminator segment were SEQ ID NO:28 and SEQ ID NO:29. The primers for detecting the correct joining of the gentamicin-NOS terminator segment and the A. thaliana downstream homologous region were SEQ ID NO:30 and SEQ ID NO:31. The primers for detecting the correct joining of the A. thaliana downstream homologous region and pCB301 were SEQ ID NO:32 and SEQ ID NO:33.

In the case of the pCB301-upstream homologous region junction, a band of 186 bp was detected, as expected. In the case of the upstream homologous region-SUC2 promoter junction, a band of 262 bp was detected, as expected. In the case of the SUC2 promoter-apoptin coding sequence junction, a band of 226 bp was detected, as expected. In the case of the apoptin coding sequence-nos terminator junction, a band of 220 bp was detected, as expected. In the case of the nos terminator-SUC2 promoter junction, a band of 172 bp was detected, as expected. In the case of the SUC2 promoter-gentamicin coding sequence junction, a band of 155 was detected, as expected. In the case of the junction of the gentamicin coding sequence-nos terminator segment and the downstream homologous region, a band of 109 bp was detected, as expected. In the case of the downstream homologous region-pCB301 junction, a band of 101 bp was detected, as expected. Therefore, all of the expected junctions were present in the pSArPP plasmid, confirming that the nine segments had been combined in the proper order. The nucleotide sequence of pSArPP is SEQ ID NO:35.

### Example 9

### Prophetic Example

A. thaliana plants are transformed with pSArPP according to the stem dip protocol, which is a modification of well known floral dip and leaf dip protocols. Health A. thaliana plants are grown until they reach the flowering stage. Optionally, the first bolts may be clipped to encourage proliferation of many secondary bolts. Plants are ready roughly 4-6 days after clipping. Optimally, plants have many immature flower clusters and only a few fertilized siliques.

The plasmid pSArPP is transformed into an appropriate Agrobacterium tumefaciens strain, such as by electroporation or a freeze-thaw method, both of which are well known in the art. The transformed bacteria may be grown on YEB or LB plates containing appropriate antibiotics in a 28° C. incubator. A colony is selected and the bacteria are resuspended in 10 μl of water. Half of this resuspension is immediately plated onto a YEB plate containing suitable antibiotics and incubated at 28° C. for 2-3 days, and the other half is used to verify the presence of the pSArPP by PCR analysis.

Bacteria from the densely grown plate described in the previous paragraph are collected by scraping them from the surface of the plate, then they are resuspended in 33 ml of YEB in a sterile tube. The OD₆₀₀ should be about 2.0. About 1.5 ml of the resuspended bacteria is then pelleted by centrifugation, then the pellet is resuspended in 1.0 to 1.2 ml of transformation buffer (MS plant salt mixture, Gamborg's vitamin solution, 5% (w/v) sucrose, pH 5.7 as adjusted with 1 N KOH). Adjustment of the OD₆₀₀ is not required. Optionally, 5% (w/v) sucrose solution can be used instead of transformation buffer; just before inoculation add Silwet L-77 to a concentration of 0.02% (v/v), and immediately mix well. If using transformation buffer, it is optional to add 0.01 mg/ml of 6-benzylaminopurine (BAP) just before transformation.

Per each transformation, 120 ml of 5% sucrose solution containing 0.03% of Silwet L-77 (Lehle Seeds) is prepared and poured into a disposable plastic bag, and then the bacteria are added. Immediately before transformation, the main stem is clipped down 1-3 inches. The inflorescences of the plants are dipped into the bacterial mixture for 10 seconds with gentle agitation. A film of liquid coating the plants is observable. The bacteria are distributed to all plant parts including very young flower shoots by gently pressing the outside of the bag by hand.

Five μl of bacterial inoculum is placed in the stem. Each plant is inoculated with 30-50 μl of bacterial inoculum.

The dipped plants are placed under a lid or cover for 16 to 24 hours to maintain high humidity. The plants can be placed on their sides if necessary. The plants are not exposed to excessive sunlight to avoid high temperature under the lid.

The plants are watered and grown as normal, tying up loose bolts with wax paper, tape, stakes, twist-ties, or by other means. Water is withheld from the plants as the seeds become mature. The dry seeds are then harvested.

Transformants are selected using an appropriate antibiotic, in this case gentamicin. Gentamicin-resistant transformants can then be screened for expression of apoptin. Gentamicin-resistant plants are expected to express apoptin.

The subject matter claimed is:

1. A plasmid comprising segments operationally configured adjacent to each other in the following order:
a binary vector comprising a broad-host-range replication origin, a gene encoding antibiotic resistance in bacteria, T-DNA border repeats configured for mediating transfer of DNA from Agrobacterium tumefaciens into a selected plant, and a multiple cloning site positioned between the T-DNA border repeats;
an upstream homologous region for targeting, together with a downstream homologous region, incorporation of DNA transferred from Agrobacterium tumefaciens into the genome of the selected plant;
a first promoter for promoting transcription of a first adjacent coding sequence;
the first adjacent sequence;
a first terminator for signaling termination of transcription of the first adjacent sequence;
a second promoter for promoting transcription of a second adjacent sequence;
the second adjacent sequence;
a second terminator for signaling termination of transcription of the second adjacent sequence; and
the downstream homologous region for targeting, together with the upstream homologous region, incorporation of DNA transferred from Agrobacterium tumefaciens into the genome of the selected plant.

2. The plasmid of claim 1 wherein the first adjacent sequence comprises a chicken anemia virus apoptin coding sequence.

3. The plasmid of claim 1 wherein the binary vector comprises pCB301.

4. The plasmid of claim 1 wherein the selected plant comprises Arabidopsis thaliana.

5. The plasmid of claim 1 wherein upstream homologous region and the downstream homologous region comprise Arabidopsis thaliana genomic DNA.

6. The plasmid of claim 1 wherein the first promoter and the second promoter comprise an Arabidopsis thaliana SUC2 promoter.

7. The plasmid of claim 1 wherein the first terminator and the second terminator comprise a nopaline synthase terminator.

8. The plasmid of claim 1 wherein the second adjacent coding sequence comprises a marker for antibiotic resistance in the selected plant.

9. The plasmid of claim 1 wherein the selected plant comprises Arabidopsis thaliana, the binary vector comprises pCB301, upstream homologous region comprises Arabidopsis thaliana genomic DNA, the first promoter comprises an Arabidopsis thaliana SUC2 promoter, the first adjacent coding sequence comprises a chicken anemia virus apoptin coding sequence, the first terminator comprises a nopaline synthase terminator, the second promoter comprises an Arabidopsis thaliana SUC2 promoter, the second adjacent coding sequence comprises a gentamicin resistance marker, the second terminator comprises a nopaline synthase terminator, and the downstream homologous region comprises Arabidopsis thaliana genomic DNA.

10. The plasmid of claim 1 wherein the first adjacent sequence comprises a multiple cloning sequence.

11. A plant expressing chicken anemia virus apoptin.

12. The plant of claim 11 wherein the plant is Arabidopsis thaliana.

13. The plant of claim 11 wherein the plant is selected from pea, corn, rice, sweet potato, rhubarb, chick pea, and tomato.

14. The plant of claim 11 wherein the plant was transformed according to a stem dip protocol.

15. A method for treating cancer comprising administering a plant part comprising apoptin to a patient in need thereof.


SEQUENCE LISTING (35 sequences)

SEQ ID NO: 1 - DNA, 31 bp, Artificial Sequence
  Forward primer for PCR amplification of pCB301.
ataacgatcacagtacattaaaaacgtccgc

SEQ ID NO: 2 - DNA, 27 bp, Artificial Sequence
  Reverse primer for PCR amplifcation of pCB301.
tgtcagttatcagattgtcgtttcccg

SEQ ID NO: 3 - DNA, 34 bp, Artificial Sequence
  Forward primer for amplification of an upstream homologous region of A. thaliana and linking to pCB301.
acgacaatctataactgacattgtcctactgatg

SEQ ID NO: 4 - DNA, 32 bp, Artificial Sequence
  Reverse primer for amplification of an upstream A. thaliana homologous region and linking to the SUC2 promoter located upstream of the apoptin coding sequence.
tatttataaaaagtttaggcctaagttatggc

SEQ ID NO: 5 - DNA, 58 bp, Artificial Sequence
  Forward primer for amplification of the SUC2 promoter.
gcctaaactttttataaataaaatctggtttcatattaatttcacacacccaagttac

SEQ ID NO: 6 - DNA, 56 bp, Artificial Sequence
  Reverse primer for amplification of the SUC2 promoter.
gagcgttcatatttgacaaaccaagaaagtaagaaaaaaaagatttctttgagagg

SEQ ID NO: 7 - DNA, 30 bp, Artificial Sequence
  Forward primer for amplification of the apoptin coding sequence.
tttgtcaaatatgaacgctctccaagaaga

SEQ ID NO: 8 - DNA, 33 bp, Artificial Sequence
  Reverse primer for amplification of the apoptin coding sequence.
ttgaacgatcttacagtcttatacgcctttttg

SEQ ID NO: 9 - DNA, 32 bp, Artificial Sequence
  Forward primer for amplification of the NOS terminator.
aagactgtaagatcgttcaaacatttggcaat

SEQ ID NO: 10 - DNA, 36 bp, Artificial Sequence
  Reverse primer for amplification of the NOS terminator.
tatttataaatcgaattcgatctagtaacatagatg

SEQ ID NO: 11 - DNA, 58 bp, Artificial Sequence
  Forward primer for amplication of the SUC2 promoter.
tcgaattcgatttataaataaaatctggtttcatattaatttcacacacccaagttac

SEQ ID NO: 12 - DNA, 56 bp, Artificial Sequence
  Reverse primer for amplification of the SUC2 promoter.
tgccattcatatttgacaaaccaagaaagtaagaaaaaaaagatttctttgagagg

SEQ ID NO: 13 - DNA, 31 bp, Artificial Sequence
  Forward primer for amplification of the gentamicin-NOS terminator.
tttgtcaaatatgaatggcagaagagaaagg

SEQ ID NO: 14 - DNA, 36 bp, Artificial Sequence
  Reverse primer for amplification of the gentamicin-NOS terminator.
agtagtattatcgaattcgatctagtaacatagatg

SEQ ID NO: 15 - DNA, 38 bp, Artificial Sequence
  Forward primer for amplication of the A. thaliana downstream homolgous region.
tcgaattcgataatactacttgttgaagtgtacattac

SEQ ID NO: 16 - DNA, 30 bp, Artificial Sequence
  Reverse primer for amplication of the A. thaliana downstream homologous region.
taatgtactgtgatcgttatctgcttcagc

SEQ ID NO: 17 - DNA, 366 bp, Chicken anemia virus
atgaacgctctccaagaagatactccacccggaccatcaacggtgttcaggccaccaacaagttcacggccgttggaaacccctcactgcagagagatccggattggtatcgctggaattacaatcactctatcgctgtgtggctgcgcgaatgctcgcgctcccacgctaagatctgcaactgcggacaattcagaaagcactggtttcaagaatgtgccggacttgaggaccgatcaacccaagcctccctcgaagaagcgatcctgcgacccctccgagtacagggtaagcgagctaaaagaaagcttgattaccactactcccagccgaccccgaaccgcaaaaaggcgtataagactgtaa

SEQ ID NO: 18 - DNA, 21 bp, Artificial Sequence
  Forward primer for detecting correct joining of pCB301 and the A. thaliana upstream homologous region by PCR.
acgctcttttctcttaggttt

SEQ ID NO: 19 - DNA, 21 bp, Artificial Sequence
  Reverse primer for detecting correct joining of pCB301 and the A. thaliana upstream homologous region by PCR.
atacatgaccaataaacgcca

SEQ ID NO: 20 - DNA, 23 bp, Artificial Sequence
  Forward primer for detecting correct joining of the A. thaliana upstream homologous region and the SUC2 promoter by PCR.
gtctctaacatatgaggatctgg

SEQ ID NO: 21 - DNA, 22 bp, Artificial Sequence
  Reverse primer for detecting correct joining of the A. thaliana upstream homologous region and the SUC2 promoter by PCR.
gtaaatgtctcccacaaaacaa

SEQ ID NO: 22 - DNA, 21 bp, Artificial Sequence
  Forward primer for detecting correct joining of the SUC2 promoter and the Apoptin coding sequence by PCR.
tacctctccactcttcctctt

SEQ ID NO: 23 - DNA, 21 bp, Artificial Sequence
  Reverse primer for detecting correct joining of the SUC2 promoter and the Apoptin coding sequence by PCR.
taattccagcgataccaatcc

SEQ ID NO: 24 - DNA, 21 bp, Artificial Sequence
  Forward primer for detecting correct joining of the Apoptin coding sequence at the NOS terminator by PCR.
aagcttgattaccactactcc

SEQ ID NO: 25 - DNA, 25 bp, Artificial Sequence
  Reverse primer for detecting correct joining of the Apoptin coding sequence and the NOS terminator by PCR.
gactctaatcataaaaacccatctc

SEQ ID NO: 26 - DNA, 22 bp, Artificial Sequence
  Forward primer for detecting correct joining of the NOS terminator and the SUC2 promoter by PCR.
gtttttatgattagagtcccgc

SEQ ID NO: 27 - DNA, 21 bp, Artificial Sequence
  Reverse primer for detecting correct joining of the NOS terminator and the SUC2 promoter by PCR.
agaaagtaacttggtgtgtga

SEQ ID NO: 28 - DNA, 21 bp, Artificial Sequence
  Forward primer for detecting correct joining of the SUC2 promoter and the gentamicin coding sequence by PCR.
aaatacctctccactcttcct

SEQ ID NO: 29 - DNA, 22 bp, Artificial Sequence
  Reverse primer for detecting correct joining of the SUC2 promoter and the gentamicin coding sequence by PCR.
gctacgtactcattctcttttc

SEQ ID NO: 30 - DNA, 20 bp, Artificial Sequence
  Forward primer for detecting correct joining of the Gentamicin-NOS terminator and the A. thaliana downstream homologous region by PCR.
aatatagcgcgcaaactagg

SEQ ID NO: 31 - DNA, 20 bp, Artificial Sequence
  Reverse primer for detecting correct joining of the Gentamicin-NOS terminator and the A. thaliana downstream homologous region by PCR.
ggtgtgatcgacaggtaatg

SEQ ID NO: 32 - DNA, 20 bp, Artificial Sequence
  Forward primer for detecting correct joining of the A. thaliana downstream homologous region and pCB301 by PCR.
tttgacagttaccttcgctg

SEQ ID NO: 33 - DNA, 21 bp, Artificial Sequence
  Reverse primer for detecting correct joining of the A. thaliana downstream homologous region and pCB301 by PCR.
tgtggtgtaaacaaattgacg

SEQ ID NO: 34 - DNA, 420 bp, Artificial Sequence
  Fusion of the gentamicin coding sequence and the NOS terminator.
atgaatggcagaagagaaaggatgagaaaagagaatgagtacgtagcttatttgcaaaggctataccccgagctccagattaactctgtttacattaacgagataggtcaaaataatgataaaaaagccttcgaggcaggaattgctcagtatgtttgagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaattcga

SEQ ID NO: 35 - DNA, 20772 bp, Artificial Sequence
  Complete sequence of the pSArPP plasmid.
cagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagccagcgctttactgagatctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgataactgacattgtcctactgatgggattgtttatacacaagtgaataaatggtcaaagaaaaatggataatgtactttggattggcgtttattggtcatgtatagacttgtttattggtataaaactaccatgtgaatcattagataaccatatggactacattttttggactgtcatcaatggacaacaactatgtctttgatttgctgtgaatgtgtttatagctcggtaaattagcactttcttttgtttgcgcattgtacaatgatacatcatatggtcataactctaaccatttacaagaaaattgtaattgatccctacaactatatggtcaacattgaatggtcgctagtggatggctacttgtaaagggttagaatataaacgcaaagcaccgtagtggacatagtttttgaaattggtggttattacttatgctttttatagaaattgtgtattgtgattctacaacggtgtagacatggttgtatggacttggggtgatggatatgcttagtaaacaagattccgtatgaacacatttgaaatggacacattattatggatttgtaagaataacaaatgcagagaaaagaatacgtgaaagaaagcatgttaataagattttgggttttagttggtatggttttgtctttatatgaaagcatgtttgatgggtctcgttcttttgtgtaagtaatgtattgttattgtggatactgtgttgaataagaaaaagacacatgatttgttttgactaagttgtgattcttgcatcaccatgacactctttttcattcgatagaaaattgatgaaagctctacacatgaatcatttgtgttcaagtgtttgtcatgtttcaccattactccacatgttaaagtttaattggacaagttataatggatacaaactatcattaactaattgggttttgaaggttgctatttgaaagcaatgtcaacatattgttcttggggcttggagacctataggattattagtttatacataatgaaatcgtgttggatgctttgatagtttgtgtccatgaaaacttgtccaacaagatgtgaccatgtggaaatgatttattctcatgtgcgaagtaaagtgctcttagcttagaggttcaacgaaacatcaatttatcaagctagaatcaaatggtaccaaacaagctgatcatatggatatgaaattcaatactggaatttgccaatctggtgctccatggaatagaacggagtaacgaggatctcgcatctccgtaaccccgtaatatcgaggttactccgaacaatttttgttctcacggaatctcttccatgtacgctgccatttcttcttctttttcgggagagagtttaaattctcaggtggaatttcaaaatccaaattaagtaagtcgagtaacgtctcacgttacgttatcaccgcctctctcgtcgccttcttgcaacagatctgaaacaaaggagagtaaggaaagaggaactttcgatctgcaaccgatctcatcgtcttcacaagcacaatttcgcaaatatgaatcagatatttacacaatctaacaaaactctaaattctattcatagaattgaagaatcatcgcaaaagctggatcgattggttgtaagattgagggaacaaatccttttcttctttcacctctagatgttattgtctgctacactcaagcttccgccgttagtcacaagcgctgccgctccgaaaccaatcgaataagcctaatcgtcacttaaatctagagaggcttctccgtaaaggaaaatacagaagctacacgattttgacaaagtctctcatgtttgattttgcaaggaaagactgaaagataaagaaagagatagatagagaaagaaaagaagaagaagagtcgatgaagaagaaaaaaagtagaccatgaagaagacgaagaaaaaaagtacaaatagagaaataatattcattatacttttttaagtaattaaattatatatataatctgataatagttacgactttcatccaacgatccactcatcatataccaacttccaaacgctaaagtattttcgttttcacttttaggggcaaatttgtcataaattcgataaaaaagttgaaaaagtgtctcatctctgcaaagtgcctcaaatggaataaaattctctaaaagtgcttaaacgtgtcacattcccttaaataaaaagatgcgataagaaggaaaaaaaaacaataaaatctgtaagtagttaaacaaaaatttacaataaaaagacacaaccgttcataagtaaaaatatattacgctaaaaagacacaattataattaaataaccgtaaataatttaacaaaaaattgcgataaaaacatatcaattcagaaatgcaaaacattacgataaaaagacgcaactcaaaaaaagatataagcgtaaatatttcaatgaaaagatgcgataaaacaacaaaacaaaccgtaaatatttcaacaaaaatacgttaaaaagatacaaccgttcagaactgcttgtaaaaaaaaagacacaacagtaaaaaaatatcataaatatttcaacgcaaagatagataaaaatacactatcgaaaaaataaccataattattttaacgaaaattaagattaaaagatacaaacattcataagtgtttgagagaaaaaaacataatcgtaaaaaataattataaatatttcaacaaaaatacgttaaaaagatataaccgtacagaactgcttgtaaaaaaaagatacaaccataaaaaatttcataaatattttgacgcaaagatagatagaaaatactgtcaaaagattaaccgtaattattttaacgaaaagcaaaattaaaagatacaaccattcagaagtgtttaagaaaagtaaaaacaattgcaaaaaataactacaaattttttttaaaaaaatacgataaaacaccaacaaaaattattattattattagattactatttagaaatatattaacaaaagtaaaatacaataataaaaagaataaatatgattacatcgatcatgcactattaaaattaaaatatattttataaatagaaaagttattgggtttcgactaatatatttaatctttttttagttaacttgactagaaaggattcgtgtatttaactattactagatgaacaaagtttcatgtttcactattataatttcatcgatttatatcaaccgaccttgcaaattcatctttctagacttttcttcctttgttgtctgatttttttcttttatacaataaataaaagactaacaaaaaataaaaataaaataattggacacgaaacgatgagtttgccctaaaaggatgaaccacaatcacgcgtcttctcatttctagtttctcagcctcctcctccttctcttccttcttatatataaatatatccacaaaatcatctcttttgcttcgctgtttctctcattattccatctctcttatcgccgctacaaacgtttcttcttcaacctcgttcagagattatcctcccttcttttctcatcttacaaggtaaagcttcttcctttttaattgattctttgatccgatttgtaaatttagtttctttatcgattttatggattgcccacatgattatttcttagatttgattggttttgaataaaaaagagatgatttgattcgtttatgctgaattaggttttcagattgtaaatattgaaacttttctctgttctcttatgggttcttgattcttttcaagatttaggattattgttgttagatcttttgaattagtgaatcttgtgtattgaaaaggtcaagtctttgtttctcttaagcagagaatgaaacaataactttgaatatgtctttttaatacaggaagttgggcagttaaagctgctgggatcaccagttttgtcaaaagtggagtgtgaagaatcttttatgtctgaacaggcttctctctttcaatcatctatccggcttttaggagagtactgtcctatatctaggaaaaagtccactcataaagttggtggtttgcgttttgaggaggacatcacgagctgtattggatttcagtttatgcaactgagtgattatcactgcctcttctgacatcccttgtttgtttacgtttgttgaatcaagatggctttgcaaaacattggtgcctccaaccgcgatgatgccttttataggtataagatgccgaggatgatgaccaagattgaaggtagaggtaacggtatcaagactaatgtggttaacatggttgagattgctaaggccttggggagacctgctgcttatactactaagtattttggttgtgagcttggtgcacagtctaagtttgatgagaagaatgggacctcgcttgtgaatggggctcatgacacttccaaacttgcaggattattggagaactttattaagaagtatgttcagtgttatggatgtgggaatcctgaaactgagatacttatcactaagacgcagatgcttcagctgaaatgtgcggcttgtgggtttctctcggatgttgatatgagggacaagctaacgtcgtttattttgaagaatcctccagagcagaagaagtcgtctaaggacaagaagtcgatgaggagagctgagaaggaaagactcagagaaggtgaagcagctgatgaagagatgaggaagctcaagaaggaggctgctagtaagaagaaagcagcgaccacgggtacttcgaaagacaaagtgtcgaagaagaaagatcattctccacctcgaagcctgtctgatgaaaatgaccaagcagacagtgaagaagatgatgacgatgtccagtggcaaacagatacttccagggaggctgctgagaaacgtatgaaagaacagctcagcgcggtaacagctgaaatggtgatgctgtctacagttgaagaaaaaaagccagtagctgaggtgaagaaggcaccagaacaagtgcatgagaatggcaatagcaagatccctgaaaatgctcatgaaaagcttgtgaatgagataaaggagcttctgagcagtggttcatctcctacccaactcaaaacagccctagcttcaaactcagcaaacccccaggagaaaatggacgccttattctctgccctctttggaggcacaggcaagggattcgcaaaagaggtgatcaagaagaagaaatatctcttggccttgatgatgatgcaagaagaagcaggagctcctgcgcagatgggtttgcttaatggaattgaatccttttgcatgaaggcaagcgcagaggctgccaaagaagttgctcttgttatcaaaggtctttacgatgaagacattttggatgaggatgtgattgttgaatggtacaataaaggtgtcaaaagctccccggttttgaagaacgtgactccgttcatcgagtggctccagaatgctgagtctgagtccgaggaagagtgatgctgtgtttctttttctttgtgtggttcaaaatttggtctgcgtcttgtgtgtgtctttctgtctggttcagtgtgcgtcttgaataaggtaagaggacactggcaaaggttacttctacttgttaagtaacactagtcactggtctctctgttgatggtttgtttatgttttgtccctttgtatttgtcatgacttagacaacttatgttgtataagttgtatgctttggttttatggttactacttttcttagaactactgtgacgattcctttgtgtttaattcgattaaacatattaggtattgttgagggttaccaaaacagatctttgtttcaagttgatccagggttctgtttgaaagtctctaacatatgaggatctggatcgacaatttcacgatcttgtttctttcttcgactacgatctctgatttaaactgtaaattttggctatatctctcgattgatcgccataacttaggcctaaactttttataaataaaatctggtttcatattaatttcacacaccaagttactttctattattaactgttataatggaccatgaaatcatttgcatatgaactgcaatgatacataatccactttgttttgtgggagacatttaccagatttcggtaaattggtattcccccttttatgtgattggtcattgatcattgttagtggccagacatttgaactcccgtttttttgtctataagaattcggaaacatatagtatcctttgaaaacggagaaacaaataacaatgtggacaaactagatataatttcaacacaagactatgggaatgattttacccactaattataatccgatcacaaggtttcaacgaactagttttccagatatcaaccaaatttactttggaattaaactaacttaaaactaattggttgttcgtaaatggtgctttttttttttgcggatgttagtaaagggttttatgtattttatattattagttatctgttttcagtgttatgttgtctcatccataaagtttatatgttttttctttgctctataacttatatatatatatgagtttacagttatatttatacatttcagatactgatcggcattttttttggtaaaaaatatatgcatgaaaaactcaagtgtttcttttttaaggaatttttaaatggtgattatatgaatataatcatatgtatatccgtatatatatgtagccagatagttaattatttgggggatatttgaattattaatgttataatattctttcttttgactcgtctggttaaattaaagaacaaaaaaaacacatacttttactgttttaaaaggttaaattaacataatttattgattacaagtgtcaagtccatgacattgcatgtaggttcgagacttcagagataacggaagagatcgataattgtgatcgtaacatccagatatgtatgtttaattttcatttagatgtggatcagagaagataagtcaaactgtcttcataatttaagacaacctcttttaatattttcccaaaacatgttttatgtaactactttgcttatgtgattgcctgaggatactattattctctgtctttattctcttcacaccacatttaaatagtttaagagcatagaaattaattattttcaaaaaggtgattatatgcatgcaaaatagcacaccatttatgtttatattttcaaattatttaatacatttcaatatttcataagtgtgatttttttttttttgtcaatttcataagtgtgatttgtcatttgtattaaacaattgtatcgcgcagtacaaataaacagtgggagaggtgaaaatgcagttataaaactgtccaataattactaacacatttaaattatctaaaaagagtgtttcaaaaaaaattcttttgaaataagaaaagtgatagatatttttacgctttcgtctgaaaataaaacaataatagtttattagaaaaatgttatcaccgaaaattattctagtgccactcgctcggatcgaaattcgaaagttatattctttctctttacctaatataaaaatcacaagaaaaatcaatccgaatatatctatcaacatagtatatgcccttacatattgtttctgacttttctctatccgaatttctcgcttcatggtttttttttaacatattctcatttaattttcattactattatataactaaaagatggaaataaaataaagtgtctttgagaatcgaaacgtccatatcagtaagatagtttgtgtgaaggtaaaatctaaaagatttaagttccaaaaacagaaaataatatattacgctaaaaaagaagaaaataattaaatacaaaacagaaaaaaataatatacgacagacacgtgtcacgaagataccctacgctatagacacagctctgttttctcttttctatgcctcaaggctctcttaacttcactgtctcctcttcggataatcctatccttctcttcctataaatacctctccactcttcctcttcctccaccactacaaccaccgcaacaaccaccaaaaaccctctcaaagaaatcttttttttcttactttcttggtttgtcaaatatgaacgctctccaagaagatactccacccggaccatcaacggtgttcaggccaccaacaagttcacggccgttggaaacccctcactgcagagagatccggattggtatcgctggaattacaatcactctatcgctgtgtggctgcgcgaatgctcgcgctcccacgctaagatctgcaactgcggacaattcagaaagcactggtttcaagaatgtgccggacttgaggaccgatcaacccaagcctccctcgaagaagcgatcctgcgacccctccgagtacagggtaagcgagctaaaagaaagcttgattaccactactcccagccgaccccgaaccgcaaaaaggcgtataagactgtaagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaattcgatttataaataaaatctggtttcatattaatttcacacaccaagttactttctattattaactgttataatggaccatgaaatcatttgcatatgaactgcaatgatacataatccactttgttttgtgggagacatttaccagatttcggtaaattggtattcccccttttatgtgattggtcattgatcattgttagtggccagacatttgaactcccgtttttttgtctataagaattcggaaacatatagtatcctttgaaaacggagaaacaaataacaatgtggacaaactagatataatttcaacacaagactatgggaatgattttacccactaattataatccgatcacaaggtttcaacgaactagttttccagatatcaaccaaatttactttggaattaaactaacttaaaactaattggttgttcgtaaatggtgctttttttttttgcggatgttagtaaagggttttatgtattttatattattagttatctgttttcagtgttatgttgtctcatccataaagtttatatgttttttctttgctctataacttatatatatatatgagtttacagttatatttatacatttcagatactgatcggcattttttttggtaaaaaatatatgcatgaaaaactcaagtgtttcttttttaaggaatttttaaatggtgattatatgaatataatcatatgtatatccgtatatatatgtagccagatagttaattatttgggggatatttgaattattaatgttataatattctttcttttgactcgtctggttaaattaaagaacaaaaaaaacacatacttttactgttttaaaaggttaaattaacataatttattgattacaagtgtcaagtccatgacattgcatgtaggttcgagacttcagagataacggaagagatcgataattgtgatcgtaacatccagatatgtatgtttaattttcatttagatgtggatcagagaagataagtcaaactgtcttcataatttaagacaacctcttttaatattttcccaaaacatgttttatgtaactactttgcttatgtgattgcctgaggatactattattctctgtctttattctcttcacaccacatttaaatagtttaagagcatagaaattaattattttcaaaaaggtgattatatgcatgcaaaatagcacaccatttatgtttatattttcaaattatttaatacatttcaatatttcataagtgtgatttttttttttttgtcaatttcataagtgtgatttgtcatttgtattaaacaattgtatcgcgcagtacaaataaacagtgggagaggtgaaaatgcagttataaaactgtccaataattactaacacatttaaattatctaaaaagagtgtttcaaaaaaaattcttttgaaataagaaaagtgatagatatttttacgctttcgtctgaaaataaaacaataatagtttattagaaaaatgttatcaccgaaaattattctagtgccactcgctcggatcgaaattcgaaagttatattctttctctttacctaatataaaaatcacaagaaaaatcaatccgaatatatctatcaacatagtatatgcccttacatattgtttctgacttttctctatccgaatttctcgcttcatggtttttttttaacatattctcatttaattttcattactattatataactaaaagatggaaataaaataaagtgtctttgagaatcgaaacgtccatatcagtaagatagtttgtgtgaaggtaaaatctaaaagatttaagttccaaaaacagaaaataatatattacgctaaaaaagaagaaaataattaaatacaaaacagaaaaaaataatatacgacagacacgtgtcacgaagataccctacgctatagacacagctctgttttctcttttctatgcctcaaggctctcttaacttcactgtctcctcttcggataatcctatccttctcttcctataaatacctctccactcttcctcttcctccaccactacaaccaccgcaacaaccaccaaaaaccctctcaaagaaatcttttttttcttactttcttggtttgtcaaatatgaatggcagaagagaaaggatgagaaaagagaatgagtacgtagcttatttgcaaaggctataccccgagctccagattaactctgtttacattaacgagataggtcaaaataatgataaaaaagccttcgaggcaggaattgctcagtatgtttgagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaattcgataatactacttgttgaagtgtacattacctgtcgatcacaccaaaagaagaacaaacacaaagaaactagacactcgaaaatcgtcgatggtctgtaattccgtaggtgcagctgaggcaggtaccaagagtcaagaaataacttctttgttttgtttgaataataagtatgtcatggctttattctcagcaagcaatccactagattctttagagttacaactgataattttataagaggttgcaaagtgactcaaaaccaaattcttgatgttttaactgaaaattatatatttagaacaaaaataatatatattggattcaaatttataagaaaatgaattgtactagattaactaaaacttctgaaagatttaagtaaaacttactctctaaaacacagcatgaaaaaataatgggtcgtaaatacatcaaatttgctggatgtcaaaatatacattttatcaaatttgctggacacaaaatcttgatactaaagcgaaaaactcaatgaaaaatttatagaaatatttttttagtaatattttgtcgactaaaattggacaagaaaaatagatttttaagaatttttttataaaaaaaaacagaaaataaatttgaattatgattgtaattttgactaaaaaaatctgcattaatcaaactagggagaagacctgcgcttttgaggggttgtttgcttttgtagattgctgattctatgtacatttgttcgtattgtttagggtatattttgaatcttgttctataaattctaaacattttttttgttattcctttgtgtgttgttaattttatcatatgttacttctttttttttgtttaattttaatcatatgttacttatatttcaatgaatgtttttggtttagcttggacttataagttgaaacttagtgcgagcagatataaattaaagggaacccatttgttacttttttatcattttttctgaatttgtattctttcctgtcataatttaaatgtgattagatcaaccatttatttgttttgtgtaacaagcttgagctgtgtactatttgttagggtcgttttacctttaactactttgtactaggtcattacccctttgatacacaggggtttgtatctatcaattgtttaactatagttttgggtttagtaatcattttagtagctatatattgacatacggtaatgcaaaagtacaataattttcatttttgattctttttaacgtaacttttgtttaatttatattctttaaatcatatatgttgtttaagaatttgttttaataagaaaggaaaatctttaggaaccatttgctataatattagaatatgatgtattataaagttttaattactaaatatttttaaattacttttgagcaaaatttgtatttcaaatacaccattgagataactataactttaatttaaaatataaattgattaacaattgtcattaatttttatattatagtttctacttatttttattaattagcttctattgatactaagttagtctttgaaggcacatcgtattagttttacttcttcattgttgtttttgtataatgataaagataacttgttgaccaaaacaatgatattttaaaagtgtagaaacttttatgctatcgttaaaacacaaagttccaaacatgataaaagataaattttacccaacaaaaaaaataaatctaagatcttttgtaattcattaagtgatataatgatatttcttgtgttcgaacgactttaaaaacatagattttttttaaaaaaatatttggaaattcaggcgcaaaggccttatattttctagaggcgggatctacaccaggttaaattcatctcttttagagaagagatcccttccaacgtgactcgaacccagcacctccacctaaaagatttaccagtgggccaacaacatgttggtgagaacatagattttctcagcttaccaaagttgaaccaatttacttttagtttttaacaacaatcgtcgacatatttgtttatattataagaaatcatcaaacacagagtattttgtaaaaattctaccatacaatttcgccttcttaactcataaatggtattttactttcatgtcaagtttaatattccaacaacaaaatttctatttttcttggttcagtttattgtttgtttttctcttttataaataatttatttaaaaaatgaatcaaatcaagccaaaacaaattaaaccttattagaacggatactaggaagacttagagttttatatttatatgaagaaaaacactcaagttttggcagatccattttaaaacccccaactaaattattaatagataaaccctccatttaagagattctaatcaaaaatcctctcaagtaatagtcggtccatttaaaaactgccaactaaatttttaacaaaaataagccttaaataaaaaaaattaattaaaagaacccttcatttatagtttcacctaaatatatcttgtttaatcgatattttgcgttaatgcttgttaaaaaacgttaaaataaataatgatatttttaagaaacgatactaatttctaaaaatcgctatttttaatctaaaaattttaaaatcgtgtaaaatagaaacctccatccgtttcatgcttatattttatttcagatttgtttagttactataattatacatgattttgttttccattgttttatagcatatgatgcaatatcttagttatattgattttgtttttcattgttttaaattgtttcatcgtatccaaaatattttgaggctaataattgaacacaaatttgaagagaccagaaaatctaatttagcaatttcagaatttgcgtttcgaacctgaaaacaaaatgaagaaatggtatcttgttgggtgcatatggtgtagatctaatcttttgtaaccatagtattaaaccttgcataagatgagtcaacaagaagaagtaaaaccaaaaaaacaagtcaatgaaaagtttttatcgatgtatgcaacctattttttttgtattatatgttttggaatttatatgaaatttatatacgctgaaaatatcttcaaaattaaatgcttacagatgtaccataattaaatgatttaatttattaatatataaaactgttcaatgttttttttttgataatttgaatattttttttaggtaagaaaaatatttgattttgatttcatttgtgtaacgcccaaacaatccttactagtgggcctcacgtcctctctctggcctatgggtccacctttgttcgacagtcagtacaccacatttagaggttttaaagacttgttgctcaccttacaaataaccacatgacctttttccatgctttttcctcactcgcatagtttgtcgcccatcctgaaacttatctagcctaagcatgcttaactttggagttctctcaggatgtttgaccgaaaatgatacttgcatttttgtgacataggtagccaaatcaattcttttacacctctccgcatgcgccaccatattcttgaaatcgggatgttacaattcacccccactaacagatcacagcgtcctcgttgcgcaccaagaccataagccccaacggtgtgagccctgttgactccgcactcgtatgggtttggatctactaccacttgtaacgccctcaccgcccctaccagtgggcccacaacttctctcgtgcgcttgggtccacccttgtttgtcggtcagtgggccacgtccatgaggctttaaagacttgttaccgaccctacaaatcaccatatgacctttctccatgctttgtcctcactcgcatggttccaccaattagttcccggaaggttacctatcttgaaactattatagcccaagcacgcttaattgtggagttttttcaagacgtgtgaccgaaaatataagtgcactttggtgacataggtggtcaattcagttattttaaacctctctgcatgcaccttcatatgcctgaaacaaggatgttacaatttgatttaacagcaatatgatactgcatatgatactgaaatacatgttgttactatttgactgagatccaagagcatcttttttaatgggtttagaagaattataataaatcaaaacaactaattatgtaaagagtcgagctctctttagatggagctgtaggaatttgagctctctttagatggagctttaggaatttgagctctcttcagatggagctttaggaatttgagctctcttcagatggagctttaggaatttgagctctcttcagatgaagctttaggaatttgggctctttttagatggagctttaggaatttgagctctctttagatggagctttaggaatttgagagagttatgtgtcaaaccgttcattgggtgataattgtaacaaaaaaaaaatgtagctttaaatgaaagattcgtaaacagagggagtggagaaatgttttatatttttatatttttatgtattaaaaaaagataatgccaacaatcatactaatattattattataaataagtcacattttgcatgtttttttttttaacttaggttgtatatacttcggttagatagtttctaactagttaaaaattatgaagatttgtgttgaaattgtggaaatagttgttctttttttagcaccaatttcaataaaagaagtaggggaattagcaagctgtaagaaatgtaattaattgatgaaagtttccttgcattttgaacaattaacgttaaaatttaggtaacaaagataattatttttatctaaaaatgacagagaaagagagaaactcggttgacaccataagataaacttggattatcaaatatggaaaaaaagtgagaaaaagttatgacatgaaatttttatgacaacgtttagttcatatatatgtaactcaacttgatgacgaggtgtctggtattactgtcatgaggttcaatggtgggacatagatttaaacaacttcttcaacaagttaaagaatatttcttttaaagttttagatctgaccaaatatgtggagacaataaaaaaaatccaatgagcatggaataatctgaatttgggtagagtattcgggagatgagaactaagggatcaaattttgagttcagatgatgaaatgaactaagtcgttttggtagttagttcataggcttattgaactatgtttggttaaggttagtaactttaaatttattgctctgtttttaatcacataataacgaataatatagagaagatgaggaagaaagtgagaaagataacgtggaagatgaagatgaagactataacgaagaagcgaacggtgaaagtaatgatgacaacggccaaaagaaatttgataactaatatgacatgaaggaatataaaacgtaataaacaatcaatgtgaagagaaatacgaaaacataggtgttgacagccaattgaatatgagaaaataaaatggatttatgattcttaaacactattgatttttataatttttaacatggtatatttgaaaagaaacttttactttaatttaaaaaccataatcaattgtatgtattttttatcaaaatatatcaaacataccctcatgtagcgtgggttcaaaatctaataatgtatttagaatagatatagttttaggaatttgagagagttacatgtcaaccgttgattaggtgatgattgtaaaaacaaatacacgcttgtagacttttctctttgtcgattattgttctagtgataaaacatgagatttctctgatgattctgttgattaagagtaaaattaagtgtgaaaatgttgtgactgagtgttggttccgccagattcttcatgtactaaattcatttgatttcttttgcaattgggaataatgtgaaggttcaacgattggcaatggaggtttgacagttaccttcgctgaagcagataacgatca